(19) United States
(12) Patent Application Publication
(10) Pub. No.: US 2024/0400756 A1
(43) Pub. Date: Dec. 5, 2024

(54) ABA TRI-BLOCK COPOLYMER AND BIORESORBABLE IMPLANTS MADE THEREWITH

(71) Applicant: Abbott Cardiovascular Systems, Inc., Santa Clara, CA (US)

(72) Inventors: Xinhua Zong, San Jose, CA (US); Ni Ding, San Jose, CA (US); Mary Beth Kossuth, San Jose, CA (US); Erik David Eli, Redwood City, CA (US)

(21) Appl. No.: 18/643,271

(22) Filed: Apr. 23, 2024

Related U.S. Application Data
(60) Provisional application No. 63/505,290, filed on May 31, 2023.

(51) Int. Cl. C08G 64/18; A61L 17/00; A61L 17/12

(52) U.S. Cl. CPC C08G 64/183; A61L 17/005; A61L 17/12

(57) ABSTRACT

A block copolymer comprises an A block and a B block. The A block provides mechanical strength while the B block provides elasticity to the polymeric material. The block copolymer may be an ABA tri-block copolymer. The A block may include one or more of polyglycolide (PGA), polylactic acid (PLA), or copolymer thereof. The B block may include a random copolymer of (i) glycolide (GA) and/or lactide (LA), (ii) trimethylene carbonate (TMC), and (iii) ε-caprolactone (CL). The block copolymer may cover an implantable device which may be used in delivering immediate hemostasis at a puncture site in a wall of a blood vessel.

### CROSS-REFERENCE TO RELATED APPLICATIONS

This application claims the benefit of and priority to U.S. Provisional Patent Application Ser. No. 63/505,290 filed on May 31, 2023, and entitled “ABA TRI-BLOCK COPOLYMER AND BIORESORBABLE IMPLANTS MADE THEREWITH,” which application is expressly incorporated herein by reference in its entirety.

### BACKGROUND

### 1. The Field of the Invention

The present disclosure relates to polymer compositions and bioresorbable implants made therewith. In more detail, embodiments of the invention relate to a polymeric material which is an ABA tri-block copolymer and implantable devices made from and/or coated with the copolymer.

### 2. The Relevant Technology

Several diagnostic and interventional vascular procedures are now performed translumenally. As an example, percutaneous vascular procedures include a mechanical approach for thrombus disruption or removal, also known as thrombectomy. After a percutaneous vascular procedure, the medical device is removed from the vessel. Once the medical device is removed from the vessel, an opening in the vessel wall remains, leading to hemorrhage into surrounding tissues.

Generally, conventional vessel closure techniques include applying manual compression and sutures. However, if the patient utilizes anticoagulants, requires long-term bed rest (e.g., 24 or more hours), or the vessel wall opening is large, these conventional techniques may no longer be adequate. An alternative hemostasis method is to use various vessel closure devices. Examples of closure devices include the Abbott Vascular Perclose family of mechanical based vessel closure devices. Alternative hemostasis methods include other vessel closure devices. One example is a plug-based closure device where the plug may be formed of a bioabsorbable and bioresorbable polymer.

Additionally, interventional vascular procedures may include the use of vascular stents. For example, self-expanding and balloon expandable stents are often used in the iliac and coronary vasculature to reduce blood loss in the instances of (or anticipated) vessel dissection or perforation. Self-expanding and balloon expandable stents are often covered for the treatment of restenotic and in-stent restentoic lesions in arteries, thrombotic occlusion, aneurysms, and traumatic or iatrogenic vessel injuries. The nonwoven matrix cover is commonly formed of nano- or submicron fibers which resemble the native extracellular matrix. While permanently covered stents are commonly used, stent restenosis may occur, especially at the distal and proximal end of the covered stent, due to smooth muscle cell (SMC) migration and proliferation across the internal elastic lamina (IEL) to luminal surface.

The specific placement of the stent acts as a scaffold to the vessel, which supports the vessel's walls and helps prevent the reoccurrence of the blockage inside the vessel. Placement of the stent may occur utilizing a catheter as a stent deployment mechanism to deliver and position the stent at the targeted area. As an example, a typical coronary stent delivery system uses a balloon expandable release method by which the mounted stent is expanded to the vessel diameter with the assistance of a balloon. Once the stent is deployed to the target area, the balloon is deflated and withdrawn with the catheter and other delivery system elements. In some instances, a stent graft or a covered stent is used. The covered stent's functions depend on the stent design, the graft material properties, the stent/graft construction, and design of the hybrid stent graft system.

In the case of the hybrid stent and graft system, the stent functions to provide a scaffold to the impaired vessel while the graft material becomes a conduit for the blood flow. In a more specific example, in peripheral arterial disease and aortic occlusions, the covered stent facilitates reopening of the vessel lumen while also providing a barrier against restenosis. Another example includes treating aneurisms to provide a bypass and partially, or in some cases completely, exclude the sac of the aneurism from the circulatory system. In yet another example, the covered stent is used in the emergency treatment of vascular injuries such as dissections or used to prevent prophylactically in the case of an accidental tear of the vessel, where the stent plays a role in scaffolding the vessel and maintaining the patency of the vessel lumen while the graft material seals tears and reestablishes blood flow distally. When the tear occurs in the blood vessel during a percutaneous intervention, the device must be fully positioned against the vessel wall to prevent or minimize blood leakage between the outer surface of the device and the vessel inner wall until coagulation may occur.

While stent grafting is a minimally invasive procedure, complications and risks are still present. In rare occasions, the covered stent may fail to provide its intended functionality. For example, the covered stent may fail to reach the target area in order to deploy the covered stent. In other instances, stent dislodgement from the catheter delivery system may occur or there may be difficulties inflating or deflating the balloon or even deploying a self-expanding stent from the sheath. Difficulties withdrawing the delivery system may occur. In other cases, the stent may fracture, migrate, cause restenotic occlusion, or be in malapposition to the arterial wall and a source of thrombus build up. Another potential problem is that the covering may tear or become separated from the remainder of the stent.

The most common failure mechanisms of covered stents are due to failure of delivery to the target lesion, stent dislodgment, and failure to seal the perforation, which can be due to inadequacies in the polymer materials used in the device. Stent dislodgment may occur during stent delivery due to poor retention, high profile, or high advancement push force may lead to poor securement of the crimped stent onto the balloon. The retention process must secure the covered stent onto the balloon; however, the process must not damage the stent, cover, or balloon. The retention of the covered stent onto the balloon is more challenging due to the thickness from the cover material on the stent. In addition, the covering may make deployment more difficult in a self-expanding stent or lead to high deployment forces (or inability to deploy).

Linti, et al., “Development, preclinical evaluation, and validation of a novel quick vascular closure device for transluminal, cardiac and radiological arterial catherization,” J. Mat. Sci. Mat. in Medicine, 2018; 29:83, disclosed a block copolymer of GA-TMC-CL for vessel closure application. However, the authors did not disclose whether the copolymer is an ABA-type or AB-type copolymer. The authors also did not disclose whether the amorphous block is GA-TMC-CL polymer or TMC-CL polymer.

Widjaja, et al., “Triblock copolymers of ε-caprolactone, L-lactide, and trimethylene carbonate: biodegradability and elastomeric behavior,” J. Biomed. Mat. Res: Part A, 2011; 99 (1) p. 38-46, disclosed an ABA block copolymer, where A is crystalline poly (L-lactide) and B is an amorphous random copolymer of TMC and CL. This copolymer demonstrates thermoplastic elastomer properties. However, the entire polymer degrades slowly due to the presence of LA in the polymer. The soft segment of TMC and CL also degrades slowly due to the relatively hydrophobic nature of the monomers. The triblock polymer is too brittle when the molar ratio of LLA to middle block (CL-TMC) is higher than 1.

Accordingly, there is an ongoing need for improved polymeric materials that may be used in medical implants, including vessel closure devices, and covered medical devices, such as stents. The polymeric material should be flexible, strong and bioresorbable in months instead of year(s). One example application of the polymeric material is covered stents for use in many vascular procedures.

### BRIEF SUMMARY OF THE INVENTION

Disclosed herein are polymer compositions and implantable devices made therewith. The polymer compositions are ABA tri-block copolymers with an A block that is crystalline and advantageously provides mechanical strength and a B block that is amorphous and advantageously provides elasticity, flexibility, and an overall relatively fast degradation rate. Thus, the ABA tri-block copolymers are biodegradable, biocompatible, and bioabsorbable and therefore suitable for use in making a variety of medical devices.

In some embodiments, the techniques described herein relate to an implantable device which includes an implantable device body and a polymeric material applied to and/or that forms the implantable device body. In some embodiments, the implantable device may be a vessel closure device that provides rapid hemostasis at a puncture site in a wall of a blood vessel. In other embodiments, the implantable device may be a stent which is covered with the polymeric material. In other embodiments, all or portions of digestive tract devices, medical patches and films, valves, clotting devices, atrial appendages, transplants, septal occluders, abdominal aortic aneurysm (AAA) repair devices, and combinations or modifications thereof can be formed of and/or coated with the polymeric material.

In some embodiments the A block of an example ABA tri-block copolymer is a polyglycolide (PGA), also known as polyglycolic acid. In some embodiments, the B block of the example ABA tri-block copolymer includes an amorphous random copolymer of glycolic acid (GA), trimethylene carbonate (TMC), and ε-caprolactone (CL). In some embodiments the A block of an example ABA tri-block copolymer may also include poly-L-Lactic Acid (PLLA), also known as poly-L-Lactide, in place of or in addition to PGA. In some embodiments, the B block of the example ABA tri-block copolymer includes an amorphous random copolymer of glycolic acid (GA) and/or lactic acid (LA), where LA can be LLA and/or DLLA. As noted, trimethylene carbonate (TMC), and ε-caprolactone (CL) can also be present. Due to its crystallinity, the A block provides mechanical strength, and the B block, being amorphous, provides elasticity. However, because it was found that TMC and CL degrade slowly, including GA/LA in the B block advantageously make more tunable degradation profiles of implantable device based on needs of targeted therapeutic applications.

In some embodiments, each A block in the ABA tri-block copolymers can have a weight percent ranging from about 10% to about 35% of the copolymer, or about 15% to about 25%, with the two A blocks together making up about 20% to about 70%, or about 30% to about 50% by weight of the copolymer. In some embodiments, the B block in the ABA tri-block copolymers can have a weight percent ranging from about 30% to about 80%, or about 50% to about 70%, by weight of the copolymer.

In some embodiments, the ABA tri-block copolymer can include the three or more monomers (i.e., GA, LA, TMC and CL) in a weight ratio of about 50/25/25 (GA/LLA:TMC:CL), where GA/LLA stands for either GA or LLA (or combination thereof). In other embodiments, the three monomers in the ABA tri-block copolymer can have a weight ratio of about 60/20/20 (GA/LLA:TMC:CL). The weight ratio can be in one or more ranges that include the foregoing weight ratios.

In some embodiments, the B block can include three or more monomers (i.e., GA, LLA, DLLA, TMC and CL) in a weight ratio of about 15/20/25 (GA/LA:TMC:CL), or about Oct. 20, 2020 (GA/LA:TMC:CL), or about 15/25/25 (GA/LA:TMC:CL), or about 30/20/20 (GA/LA:TMC:CL), GA/LA stands for either GA or LA (LLA or DLLA), or a combination thereof. The weight ratio can be in one or more ranges that include the foregoing weight ratios. In some embodiments, the soft segment contains no or is substantially free of LLA and/or DLLA, but contains or consists of or consists essentially of the three monomers of GA, TMC and CL. In another embodiment, the soft segment contains no or is substantially free of GA, but contains or consists of or consists essentially of LLA and/or DLLA, TMC and CL.

The ABA tri-block copolymer can have first and second glass transition temperatures (Tg-1 and Tg-2) for the amorphous phase and a melting temperature (Tm) and enthalpy of fusion (ΔH) for the crystalline phase.

In some embodiments, Tg-1 can be in a range of about −30° C. to about 0° C., or about −20° C. to about −5° C., and Tg-2 can be in a range of about 30° C. to about 55° C., or about 30° C. to about 40° C.

In some embodiments, Tm can be in a range of about 110° C. to about 200° C., or about 145° C. to about 200° C., and ΔH can be in a range of about 5 J/g to about 50 J/g, or about 10 J/g to about 35 J/g.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

A description of various aspects and features of the invention will be rendered by reference to various representative embodiments thereof illustrated in the appended drawings. It is appreciated that these drawings depict only typical embodiments of the invention and are therefore not to be considered limiting of its scope.

FIGS. 1A and 1B illustrate examples of medical devices made from an ABA polymeric material according to an embodiment of the present invention.

FIGS. 2A through 2F illustrate an exemplary covered medical device made with an ABA polymeric material according to an embodiment of the present invention.

FIG. 3 illustrates an attachment of a graft to a stent or scaffold according to an embodiment of the present invention.

FIG. 4 illustrates an attachment of a graft to a stent or scaffold according to an embodiment of the present invention.

FIG. 5 illustrates an attachment of a graft to a stent or scaffold according to an embodiment of the present invention.

FIGS. 6A through 6C illustrate thermograms of various exemplary triblock copolymers by differential scanning calorimetry (DSC) at a first heat.

FIGS. 7A through 7C illustrate DSC thermograms of the same triblock copolymers as FIGS. 6A-6C, at a second heat.

FIG. 8 illustrates a stress strain measurement of an exemplary triblock copolymer.

FIG. 9 illustrates molar mass change over time for various exemplary triblock copolymers.

FIG. 10 illustrates mass loss change over time for various exemplary triblock copolymers.

### DETAILED DESCRIPTION

One or more specific embodiments of the present disclosure will be described below. To provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure generally relate to ABA tri-block copolymers. The ABA tri-block copolymers disclosed herein includes an A block, which is selected to primarily provide or contribute to mechanical strength, and a B block which is selected to primarily provide or contribute flexibility and elasticity. The polymer compositions may be bioabsorbable, biocompatible, and biodegradable.

One or more embodiments of the present disclosure may generally relate to apparatuses, systems, and methods of making and using an implantable medical device including a polymeric material (e.g., a closure device or covered stent), that uses an ABA tri-block copolymer. The implantable medical device may be used to provide immediate or substantially immediate hemostasis at a vessel wall opening. Additionally, the implantable medical device may be used as a covered stent where the stent provides scaffolding to the vessel and the polymeric material is used as a covering and/or coating. In this instance, the polymeric material is both bioabsorbable and biocompatible and degrades and/or is reabsorbed after the stent has been properly placed and secured. The disclosed polymeric material in the implantable medical device provides a balance of crystalline hard segment to withstand force from the medical procedure and an amorphous soft segment providing delivery, expandability, and conformity with vessel walls. Therefore, the implantable medical device is appropriate to use in many interventional vascular procedures.

While the present disclosure will describe a particular implementation of apparatuses and systems, with associated methods, for an implantable medical device for use in interventional vascular procedures, it should be understood that any of the systems, apparatuses, and methods described herein may be applicable to other uses, including and not limited to digestive tract devices, medical patches and films, valves, clotting devices, atrial appendages, occluders, AAA repair, AV fistulas, tissue transplants and/or a coronary sinus reducer for use in angina treatment. Additionally, elements described in relation to any embodiment depicted and/or described herein may be combinable with elements described in relation to any other embodiment depicted and/or described herein.

### Polymeric Material

It is desired that some embodiments of the polymeric material provide the following characteristics. In some embodiments, the polymeric material can be moldable to be appropriately shaped. For example, in the case of a balloon expandable covered stent, the polymeric material is a woven or non-woven mesh which is incorporated around the stent and should be sufficiently flexible to allow the balloon expandable covered stent to expand. In some embodiments, the polymeric material may be molded and allow moldability. In some embodiments the polymeric material can be compressible for appropriate delivery. In the particular case of a plug-based closure device, the polymeric material is advantageously able to push through the vessel wall prior to the implantable device being used. Therefore, the polymeric material will desirably have compression properties. In some embodiments, the polymeric material may be soluble for spray coating or dip-coating on the stent or other devices. Roller-coating and/or ink-jetting are also possible. Such coating may contain one or more anti-proliferative drugs, such as sirolimus (Rapamycin), everolimus, zotarolimus, deforolimus, μmirolimus, temsirolimus, and its analog or antimicrotubule agents such as paclitaxel. In yet other embodiments, the polymeric material may be electrospun to create a filament like structure.

In some embodiments the polymeric material can be elastic. The elastic properties allow the polymeric material to fit the particular vessel's geometry, such as in the use of a vessel closure device or around a stent used as vessel scaffolding, or a stent coating which can tolerate the stent crimping and expansion. In some embodiments, the polymeric material should be able to undergo low deformation during the deployment. In other embodiments, the polymeric material should be able to undergo low deformation after the deployment. In even other embodiments, the polymeric material should be able to undergo low deformation both during and after deployment. In the case of a covered stent, the polymeric material can have low deformation while the covered stent is being inserted and continue to have low deformation until the polymeric material begins to degrade, leaving the stent in the appropriate position. In some embodiments, the polymeric material can have a low deployment force.

In some embodiments, the polymeric material can have relatively fast degradation following implantation. For example, the polymeric material may degrade in less than 1month, within 2 months, within 3 months, within 4 months, within 5 months, within 6months, within 7 months, within 8 months, within 1 year, or more than 1 year following implantation. In some procedures, it may be preferable for the polymeric material degrade within about 1 month to about 6 months following implantation. In some embodiments, the polymeric material should be biocompatible, or bioabsorbable, or bioresorbable, or biodegradable, or a combination thereof. For example, when the polymeric material is used in a covered stent, the polymeric material should be biocompatible with the vessel into which the stent is being inserted. As the polymeric material degrades, the degraded material is advantageously bioabsorbable by the vessel or removed through body's excretory system. As another example, when the polymeric material is utilized in a vessel closure device, the polymeric material is advantageously biocompatible with the vessel walls and bioabsorbable by the body as the vessel walls close without the polymeric material.

Turning now to specific embodiments of ABA tri-block copolymers, the polymers include an A block and a B block. The A block is selected primarily to provide or contribute mechanical strength, while the B block is selected primarily to provide or contribute elasticity and flexibility, to the polymeric material.

The monomeric constituents of the ABA tri-block copolymer typically include glycolic acid (GA), which is often provided as condensed glycolide dimer, lactic acid (LA), which can also be provided as condensed lactide dimer, trimethylene carbonate (TMC), and ε-caprolactone (CL). Glycolic acid, glycolide dimer, and polyglycolide have the following chemical structures:

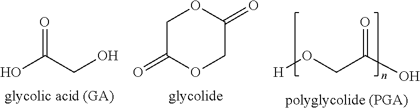

Lactic acid, lactide dimer, and polylactide (aka polylactic acid) have the following chemical structures:

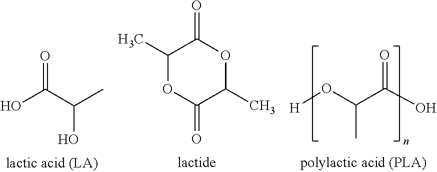

When used to form the A block, PGA will typically have n glycolide units joined to each other to form a crystalline polymer, with a terminal hydroxyl or terminal carboxylate being condensed with and forming a covalent bond with a corresponding terminal monomer of the B block. When used in the B block, a diol initiator may be used to create the glycolic acid/glycolide (GA)/lactic acid (LA), trimethylene carbonate (TMC), or ε-caprolactone (CL) polymer through anionic living polymerization.

In some embodiments, the B block is synthesized first by using a diol as an initiator and a catalyst, which yields a B block terminated with two hydroxyl groups-one at each end. In some embodiments, the catalyst may be a tin catalyst such as stannous octanoate. In these embodiments, the A block may be synthesized after the B block using the same tin catalyst.

Trimethylene carbonate (TMC) and its monomeric unit in poly (trimethylene carbonate) after ring opening and polymerization have the following chemical structures:

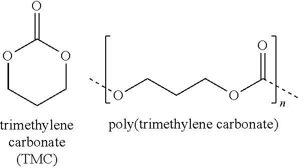

ε-caprolactone (CL) and its monomeric unit in polycaprolactone after ring opening and polymerization have the following chemical structures:

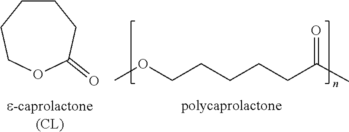

In some embodiments, the A block is formed of a crystalline polyglycolide (PGA) and/or poly (l-lactic acid) (PLLA). Polyglycolide may also be referred to as poly (glycolic acid)/polyglycolic acid (PGA). PGA is a biodegradable, thermoplastic polymer and is a linear aliphatic polyester.

In some embodiments the B block is formed of an amorphous random copolymer. The amorphous random copolymer includes glycolic acid/glycolide (GA) and/or lactic acid (LA), tri-methylene carbonate (TMC), and ε-caprolactone (CL) monomeric units. Glycolide and lactide are each typically provided in dimer form. TMC is also referred to as 1,3-propylene carbonate and is initially a 6-membered cyclic carbonate ester before ring opening and polymerization. CL is also referred to as caprolactone and is a lactone (e.g., a cyclic ester), which includes a seven-member ring initially.

The polymeric materials used to make medical implants are tri-block copolymers with an ABA symmetric triblock copolymer pattern. The copolymers exhibit a blend of mechanical strength and elasticity. The A units comprised of PGA and/or PLLA provide or contribute mainly to mechanical strength and, therefore, the A blocks play the majority role in the polymeric material's mechanical strength. The TMC and CL monomeric units in the B block provide or contribute mainly to elasticity and therefore the B block plays the majority role in the polymeric material's elasticity and flexibility. The GA and/or LA units in the B block are more hydrophilic than TMC and CL and promote faster biodegradation and resorption by the body.

As described above, it is desired in some embodiments that the polymeric material have a relatively fast degradation time. In general, the TMC and CL in the B block degrade relatively slowly. To offset this, embodiments may advantageously include GA and/or LA in the B block to accelerate degradation of the random copolymer.

In order to identify one or more ABA tri-block copolymers having suitability for an intended purpose in an implantable device, multiple compositions of ABA tri-block polymers (aka terpolymers) were created and tested to determine the optimal composition with desired properties in applications. The following tables below summarize the compositions of such example copolymers.

TABLE 1

| | A Block | B Block | | |
|---|---|---|---|---|
| TerPolymer | (Hard Segment) | (Soft Segment) | | |
| A-B-A Block | GA or LLA (%) | GA or LA (%) | TMC (%) | CL (%) |
| Example-1 | 30 | 10 | 5 | 25 |
| Example-2 | 25 | 25 | 5 | 20 |
| Example-3 | 25 | 15 | 10 | 25 |
| Example-4 | 25 | 10 | 20 | 20 |
| Example-5 | 22.5 | 25 | 5 | 25 |
| Example-6 | 22.5 | 15 | 20 | 20 |
| Example-7 | 22.5 | 10 | 25 | 20 |
| Example-8 | 20 | 30 | 5 | 25 |
| Example-9 | 20 | 20 | 20 | 20 |
| Example-10 | 20 | 15 | 25 | 20 |
| Example-11 | 17.5 | 30 | 10 | 25 |
| Example-12 | 17.5 | 20 | 25 | 20 |
| Example-13 | 17.5 | 15 | 25 | 25 |
| Example-14 | 15 | 30 | 20 | 20 |
| Example-15 | 15 | 20 | 25 | 25 |

Table 1 shows some examples of disclosed embodiments. Example embodiments may include two A blocks where each A block is less than about 10% weight, about 10% weight, about 15% weight, about 20% weight, about 25% weight, about 30% weight, or more than about 30% weight. In some embodiments, the A block may be formed of GA, LLA. Additionally, example embodiments may include a B block that contains less than about 15% weight, about 15% weight, about 17.5% weight, about 20% weight, about 22.5% weight, about 25% weight, about 30% weight, or more than about 30% weight of GA, LA, or a combination of GA and LA, less than about 5% weight, about 5% weight, about 10% weight, about 20% weight, about 25% weight, or more than about 25% weight of TMC, and less than about 20% weight, about 20% weight, about 25% weight, or more than about 25% weight of CL.

In some embodiments, the A block may contain only PGA and the B block may contain GA rather than LA, in addition to the TMC and CL, as monomeric units. In other embodiments, the A block may contain only PLLA rather than PGA and the B block may contain LA rather than GA, in addition to the TMC and CL, as monomeric units. In other embodiments, the A block may contain only PGA and the B block may only contain LA, in addition to the TMC and CL, as monomeric units. Alternatively, in yet other embodiments the A block may contain only PLLA and the B block may only contain GA, in addition to the TMC and CL, as monomeric units. In yet other embodiments, the A block may contain a copolymer of PGA and PLLA, which may impact (e.g., decrease) the crystallinity of the A block, and the B block may include only GA, only LLA, only DLLA, both GA and LLA, both DLLA and LLA, both GA and LLA, or GA, LLA, and DLLA, in addition to TMC and CL, as monomeric units. Also, in some embodiments, the A block may contain only PGA, only PLLA, or both PGA and PLLA, while the B block contains a mixture of GA, LLA, and DLLA, in addition to the TMC and CL, as monomeric units.

Depending on the application and desired functionality of the ABA tri-block polymeric material, the percentage of A block materials and B block materials can be optimized to give optimal properties. For example, Example #13 appears to be a favorable formulation for use in making the anchor seal and cap seal for closing perforated vessels due to its physical characteristics, while having good bioabsorption.

Other properties of the ABA tri-block polymeric materials may be optimized to provide functional features. For example, in some embodiments, each A block in the ABA tri-block copolymer can have a weight percent ranging from about 10% to about 30% of the copolymer, or about 15% to about 25%, with the two A blocks together making up about 20% to about 60%, or about 30% to about 50% by weight of the copolymer. In some embodiments, the B block in the ABA tri-block copolymer can have weight percent ranging from about 40% to about 80%, or about 50% to about 70%, by weight of the copolymer.

In some embodiments, the ABA tri-block copolymer can include the monomers (i.e., GA and/or LA, TMC and CL) in a weight ratio of about 50/25/25 (GA/LA:TMC:CL). In other embodiments, the monomers in the ABA tri-block copolymer can have a weight ratio of about 60/20/20 (GA/LA:TMC:CL). In yet other embodiments, the monomers in the ABA tri-block copolymer can have a weight ratio of 50-60 parts GA/LA:10-40 parts TMC:10-40 parts CL, with the proviso that the block copolymer includes 100 parts of combined GA/LA, TMC and CL. In yet other embodiments, the monomers in the ABA tri-block copolymer can have a weight ratio of 50-60 parts GA/LA:20-25 parts TMC:20-25 parts CL, with the proviso that the block copolymer includes 100 parts of combined GA/LA, TMC and CL.

In some embodiments, the B block can include the monomers (i.e., GA/LA, TMC and CL) in a weight ratio of about 15/20/25 (GA/LA:TMC:CL), or about Oct. 20, 2020 (GA/LA:TMC:CL), or about 15/25/25 (GA/LA:TMC:CL), or about 30/20/20 (GA/LA:TMC:CL), or in a weight ratio of about 15-30 parts GA:20-25 parts TMC:20-25 parts CL, or in a weight ratio of about 15-30 parts GA:10-40 parts TMC:10-40 parts CL.

The ABA tri-block copolymer can have first and second glass transition temperatures (Tg-1 and Tg-2) for the amorphous phase and a melting temperature (Tm) and enthalpy of fusion (ΔH) for the crystalline phase.

In some embodiments, Tg-1 can be in a range of about −10° C. to about −40° C., or about −15° C. to about −30° C., and Tg-2 can be in a range of about 40° C. to about 55° C., or about 45° C. to about 50° C.

In some embodiments, Tm can be in a range of about 105° C. to about 220° C., or about 145° C. to about 210° C., and ΔH can be in a range of about 10 J/g to about 35 J/g, or about 13 J/g to about 30 J/g.

For example, in some embodiments, the block copolymer can have a tensile strength in a range of about 5 MPa to about 100 MPa, preferably about 10 MPa to about 40 MPa, and more preferably about 10 MPa to about 30 MPa.

In some embodiments, the block copolymer can have a first glass transition temperature Tg in a range of about −30° C. to about 0° C., preferably about −20° C. to about −5° C., and more preferably about −15° C. to about −10° C.

In some embodiments, the block copolymer can have an elongation at break in a range of about 50% to about 1000%, preferably about 100% to about 800%, and more preferably about 200% to about 400%.

In some embodiments, the block copolymer can have an inherent viscosity in a range of about 0.5 to about 1.8 dL/g, preferably about 0.7 to about 1.5 dL/g, and more preferably about 0.9 to about 1.1dL/g.

In some embodiments, the block copolymer can have an elastic modulus in a range of about 20 MPa to about 500 MPa, preferably about 40 MPa to about 400 MPa, and more preferably about 40 MPa to about 200 MPa.

In some embodiments, the block copolymer can have a percent crystallinity in a range of about 5% to about 25%, preferably about 5% to about 20%, and more preferably about 7% to about 15%.

### Implantable Device Body

Embodiments of the invention also generally include an implantable device, which includes an implantable device body. The implantable device body may vary considerably depending on the intended application. Some example implantable device bodies will now be discussed.

In some embodiments, the implantable device body relates to a vessel closure delivery device. The vessel closure delivery device may include an actuator, an anchor, a cap, a closure element, a delivery sheath, a fluid-blocking component, and/or a suture element. Examples of the implantable device body can be found in co-pending U.S. Provisional Patent Application No. 63/495,360, filed Apr. 11, 2023, and entitled “Vessel Closure Device with Improved Safety and Tract Hemostasis” and U.S. Patent Application Publication No. 2022/0110617, entitled “Vessel Closure Device with Improved Safety and Tract Hemostasis”, the disclosures of which are incorporated herein in their entireties by reference.

In some embodiments, the implantable device body is a stent. The stent may include a balloon-expandable stent, self-expanding stent, coronary stent, peripheral stent, carotid stent, neurological stent, vascular stent, ureteral stent, prostatic stent, colon stent, esophageal stent, pancreatic stent, biliary stent, glaucoma drainage stent, stent for AV fistula, coronary sinus reducer, or other appropriate stent types. In some embodiments, the balloon expandable stent is the Omnilink Elite™ Vascular Balloon-Expandable Stent. In some embodiments, the stent may be formed of a variety of metals including cobalt chromium, stainless steel, nitinol, or other appropriate metals. In some embodiments the stent (e.g., not including the covering or coating) has a thickness ranging from about 50 μm to about 250 μm, preferably from about 90 μm to about 210 μm, more preferably from about 110 μm to about 160 μm.

In some embodiments, the implantable device body can be digestive tract devices, medical patches and films, valves, clotting devices, atrial appendages, AAA repair devices, and/or tissue transplants. The covering may be on the outside, inside, some combination thereof, or encapsulating the stent. The covering may be for the entire stent or a portion of the stent, e.g., which portion requires sealing specifically due to the purpose of that particular stent graft.

### Applying Polymeric Material Onto Implantable Device

In some embodiments, the polymeric material is applied to the implantable device body by the use of an electrospinning process. In general, electrospinning is a fiber production method based on using electric force to attract charged polymer solution threads to create a polymer fiber. The polymer fiber may differ in fiber diameter based on the electrospinning process.

In some embodiments, an ABA copolymer solution is provided and utilizes an electrospinning process to produce ABA copolymer fibers. The ABA copolymer solution flows out of a needle and is electrostatically attracted to an opposite charge on a mandrel. The polymer is wrapped around the mandrel to create nonwoven 3D web with individual ABA copolymer fibers. The final individual ABA copolymer fibers may have a spider web like consistency and appearance after the electrospinning process. Additionally, in some embodiments, the ABA copolymer fibers may be drawn out into long filaments during the electrospinning process.

In some embodiments, the electrospun polymeric material is used to create the implantable device body as well as the polymeric coating. Since the polymeric material advantageously has a rubber like form and is biodegradable, the polymeric material may be used to create some portion, most or all the product's structure. For example, the extent and location of the covering may depend on the purpose of such device. By way of further example, for anchoring one might want the covering in a middle portion of the device, but the edges may anchor in the vessel or perhaps even limit risk for edge thrombus build-up. In some embodiments, the polymeric material can be used to cover an implantable medical device, such as a stent. In some embodiments, the polymeric material can be used to form a cap and/or anchor for a vessel closure device.

In instances where the polymeric material is used as a covering (e.g., a covered stent), the electrospun filaments may be directly overlayed onto the implantable device body. In other embodiments, the electrospun polymeric filaments are initially deposited into a rope-like structure. The rope-like polymeric material is then overlayed onto the implantable device body. In this embodiment, the polymeric material may have a durable, highly clastic, tear resistant, and biodegradable structure with a consistency similar to Teflon tape (e.g., stretchy with recoverability).

In other embodiments, the polymeric material may be dissolved in a solution. The solvent may be hexafluoro isopropanol or other appropriate solvent(s). The active pharmaceutical ingredient such as everolimus may be co-dissolved with the polymer and applied on the stent as an anti-restenotic coating through spraying, roller-coating, and/or ink-jetting.

In other embodiments, the polymeric material may be melted and then be electronically sprayed as a coating onto a medical device and/or drug delivery device.

Example Implantable Devices with Polymeric Materials

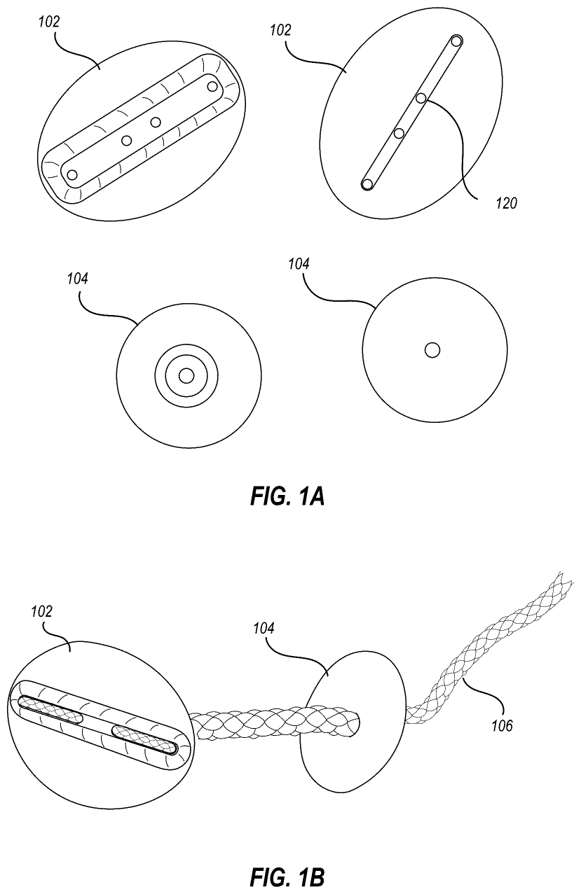

In the case of a vessel closure device, some embodiments include an anchor and a closure element, such as a cap, made from the ABA polymer. FIGS. 1A and 1B illustrate an example of an anchor 102 and a cap 104 made from the ABA polymer. The anchor 102 may be passed through an opening (e.g., puncture) defined in a wall of a blood vessel and deployed into the vessel lumen. The anchor 102 can then be drawn proximally to draw the anchor into contact with an inner surface of the blood vessel wall. The cap 104 can then be deployed on the outside surface of the blood vessel wall to close the puncture by advancing the cap 104 along the suture element 106. The suture element 106 can optionally be formed of or coated in the ABA polymer.

The anchor 102 includes a keel 120 (discussed more fully below) and a surface. As discussed elsewhere, the suture 106 can be threaded through holes or eyelets within the anchor 102 to thereby secure the suture 106 to the anchor 102, such as illustrated in FIG. 1B. Such a configuration also allows for any forces applied to the suture 106 (i.e., pulling or tensioning the suture 106) to be transferred to the anchor 102. For example, when a physician or other practitioner exerts a proximal pulling or tugging force on the suture 106, a proximal pulling or tugging force will be exerted on the anchor 102, moving the anchor 102 in a proximal direction.

The extravascular cap 104 can be made from ABA polymer and be of sufficient size and geometry to prevent it from passing through the puncture access site at the surface of the blood vessel. The size and geometry of the cap 104 can significantly increase patient safety by preventing extravascular components from passing through the access site during and/or after deployment. The cap 104 can have a diameter ranging from about 1 mm to about 10 mm, from about 3 mm to about 8 mm, from about 4 mm to about 5 mm, or a range defined by any two of the foregoing values. The cap 104 can have another size and shape based upon the specific dimensions of the access site, so that the cap 104 does not pass through the puncture/access site and into the vessel lumen.

The cap 104 can have a low-profile and be made from a biodegradable material. The cap 104 can also have a desired flexibility to conform to the anatomy at the access site (especially in vessels with significant calcification present) and provide more effective scaling than would rigid materials. The cap 104 can be deployed through an access tissue tract and placed on top of the vessel, acting as the primary extravascular seal with the vessel wall or other tissue disposed between the anchor 102 and the cap 104.

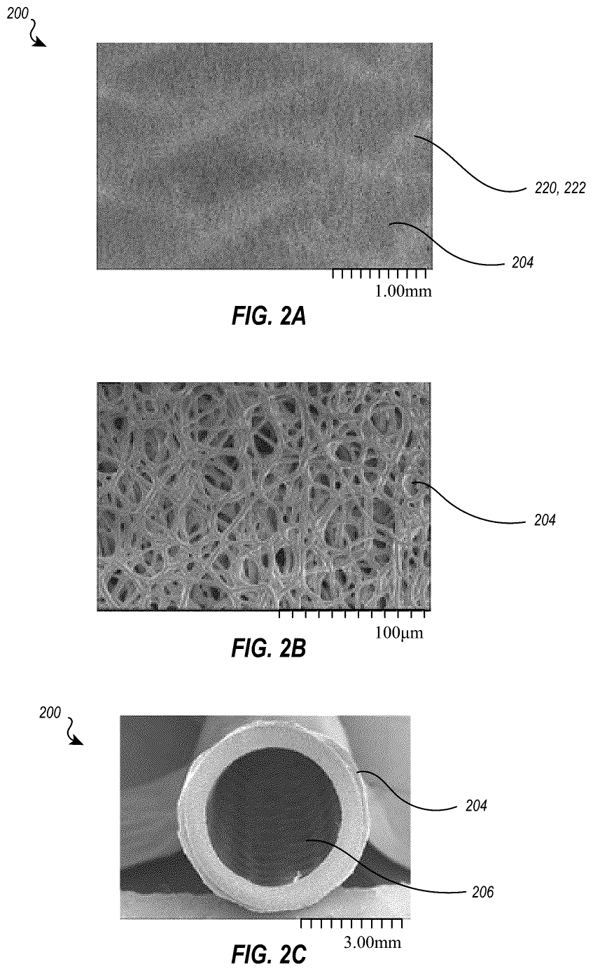

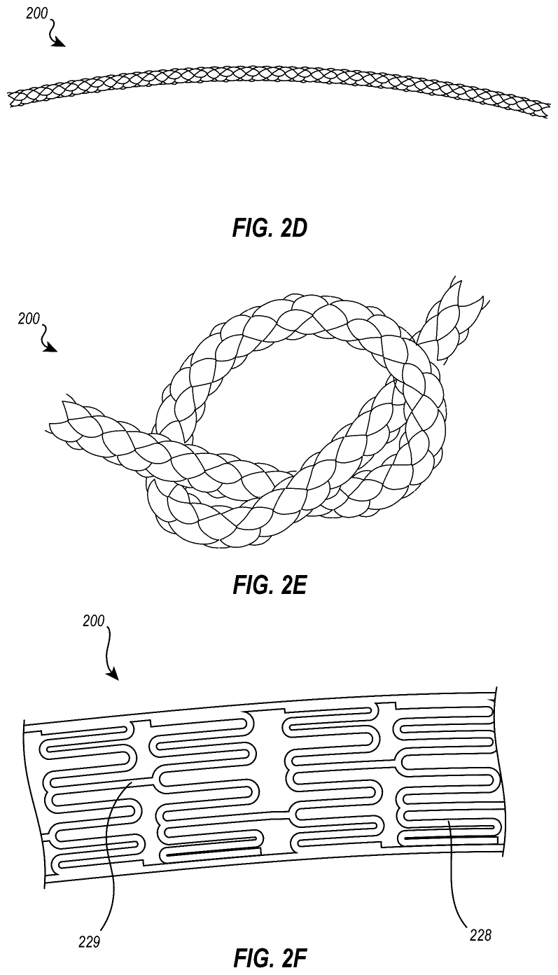

In the case of a covered stent, some embodiments include stents, grafts, or other features created from the ABA polymer. FIGS. 2A through 2F illustrate an example of a covered stent or scaffold made from the ABA polymer. FIGS. 2A and 2B are SEM images of a polymer coating 204, such as formed by the ABA polymer, on a stent or scaffold body 222 of a stent or scaffold 220 to form a covered stent or scaffold 200. FIG. 2C illustrates the covered stent or scaffold 200 with a lumen 206. FIG. 2D illustrates a profile view of the covered stent or scaffold 200, while FIG. 2E illustrates an example of the flexibility of the covered stent or scaffold 200 where the body or frame 222 and the polymer coating 204 are both formed of the ABA polymer. FIG. 2F illustrates a zoomed or more detailed view of the body or frame 222 illustrating the stent or scaffold rings 228 and connectors 229. While the covered stent or scaffold 200 of FIGS. 2A through 2F illustrate a self-expandable stent or scaffold, it will be understood that other stents or scaffolds are possible, including balloon expandable stents or scaffolds that are coated with the polymer coating 204, while the underlying stent body or scaffold body is formed of a metal alloy, such as nitinol, elgiloy, 316 stainless steel, L605 Co—Cr, MP35N cobalt alloy, ternary nickel titanium platinum, platinum cobalt, tantalum alloys, and combinations or modifications thereof.

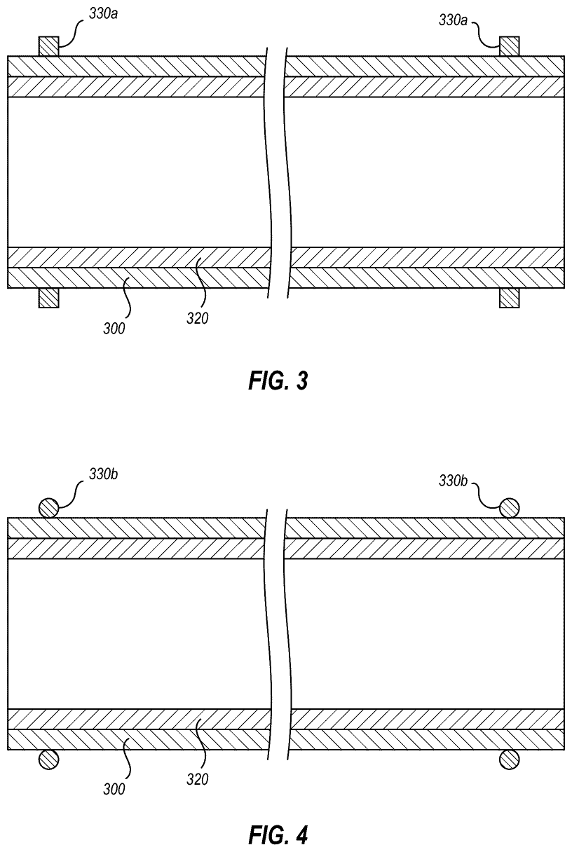

In another other configuration as illustrated schematically in FIG. 3, attachment or coupling of the graft 300 to the crimped stent or scaffold 320 can be performed using circular flat rings or ribbons 330a surrounding the ends of the graft 300 and maintaining attachment or coupling of the graft body to the body or frame at both ends. For instance, the circular flat rings or ribbons (such as O-rings) 330a may be made from the ABA polymer or other polymer such as shape memory polymer (SMP). In the case of a shape memory polymer, the shape memory polymer can be programmed such that the diameter of the circular flat rings or ribbons 330a at room temperature will be small and will subsequently enlarge to the expanded stent diameter once thermally activated. Assembly of the graft 300 and crimped stent or scaffold 320 can include positioning of the graft body onto the crimped body or frame. The circular rings or ribbons 330a can then be loaded at both the proximal and distal ends, which secures the graft 300 in place.

In another embodiment, as illustrated schematically in FIG. 4, attachment or coupling of the graft 300 to the crimped stent or scaffold 320 can be accomplished using circular wire rings 330b at both ends. The wire 330b may be constructed from a shape memory alloy such a nitinol (nickel-titanium alloy). The wire 330b may have a sinusoidal shape which can be deformed at room temperature when placed onto the graft 300 mounted or disposed over the body or frame 320. The recovered pre-deformed shape of the ring 330b may allow full extension of the stent or scaffold 320 during deployment at nominal pressure upon temperature activation. Such rings or ribbons 330a/330b may be of any desired cross-sectional shape.

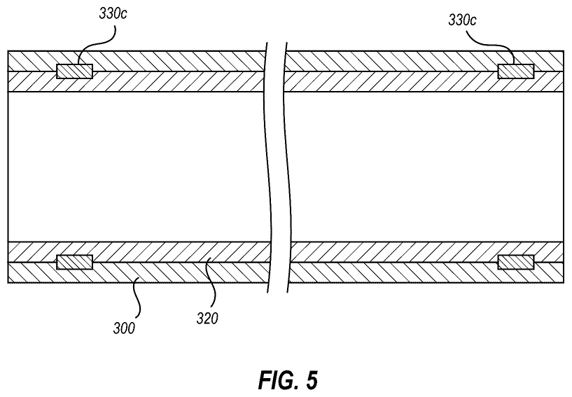

In yet other embodiments, as illustrated schematically in FIG. 5, attachment or coupling of the graft 300 to the crimped stent or scaffold 320 can be performed by spot gluing or welding 330c the graft 300 to the proximal and distal ends of the stent or scaffold 320. In other embodiments, securement of the graft 300 to the crimped stent or scaffold 320 can be performed by using an adhesive, thermal bonding, solvent bonding, laser welding technology, and combinations or modifications thereof. Following positioning the graft body onto the crimped stent or scaffold 320, and loading onto a mandrel, spot gluing or laser welding can be performed at both ends or done at specified internal locations. The welding, gluing, etc. may be fully circular or at a number of discreet non-continuous positions.

### Experimental Results

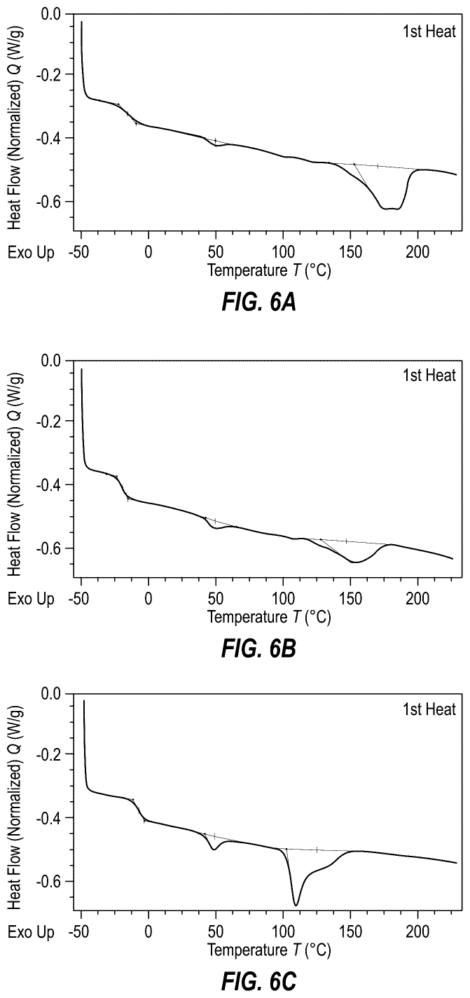

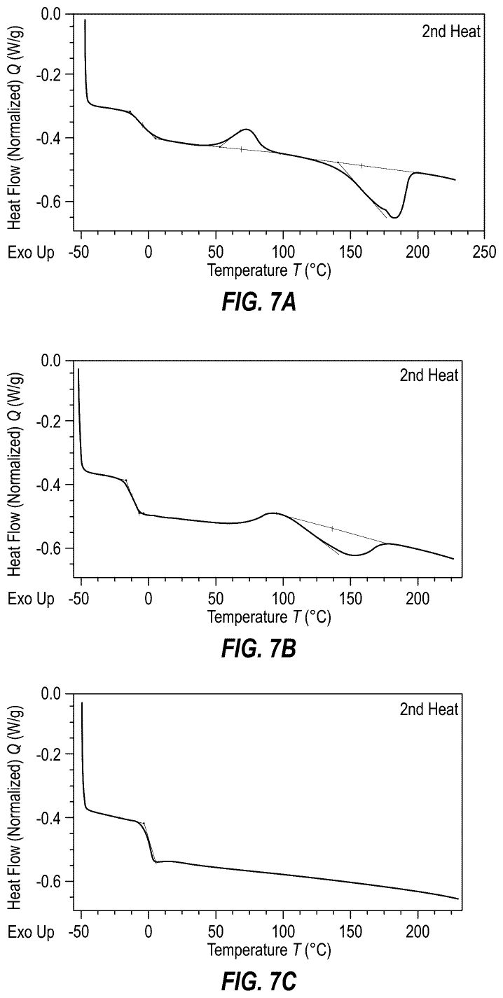

FIGS. 6A-6C and FIG. 7A-7C illustrate thermal properties of three example embodiments of an A-B-A block copolymer. Glass transition temperatures (Tg) for the amorphous phase of B-block, melting temperature (Tm) and enthalpy of fusion (ΔH) for the crystalline phase of A-block were measured by differential scanning calorimetry (DSC) at a heating rate of 10° C./min in a temperature range of −50-230° C. The samples were scanned using two heating cycles. FIG. 6 shows the first heating cycle while FIG. 7 shows the second heating cycle.

Glass transition temperatures (Tg) of amorphous B-block (soft segment) in three example embodiments of an A-B-A copolymer is in the range of −30° C. to 0° C. The melting peak of crystalline A-block (hard segment) in the three example embodiments of an A-B-A copolymer is in the range of 108° C. to 183° C. The block copolymer in FIGS. 6A and 7A shows the highest Tm at about 176-183° C., and ΔH of 26-30 J/g. The block copolymer in FIGS. 6B and 7B exhibits a Tm at about 151-155° C., and ΔH of 11-16 J/g. The block copolymer in FIG. 6C exhibits a Tm at about 108° C., and ΔH of 21 J/g. FIG. 7C illustrates no melting peak due to a slow crystallization process with the lowest content of crystalline A-block in an example embodiment of an A-B-A copolymer.

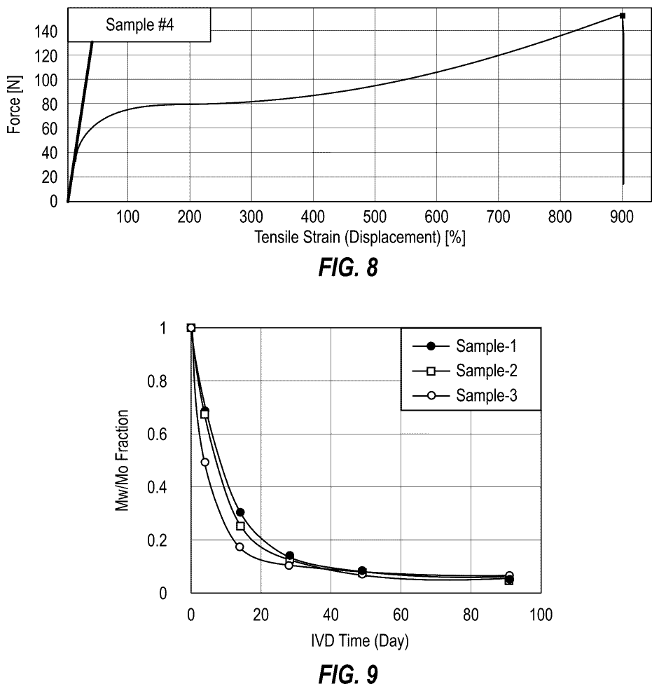

FIG. 8 illustrates mechanical properties of an example embodiment of an A-B-A block copolymer carried out using the INSTRON. The tensile strength and the maximum elongation depend on the weight ratio of the A-block as well as monomer ratios within the B-block. Sample 4 has mechanical properties with elongation up to 900% and elastomeric properties due to the absence of a measurable yield stress in the stress-strain curve.

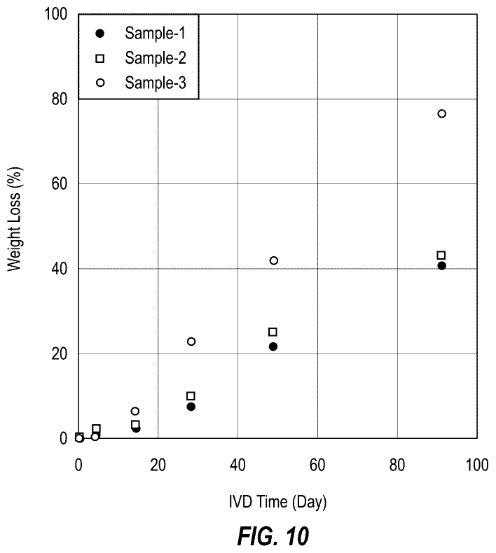

FIG. 9 and FIG. 10 illustrate in-vitro degradation properties of three example embodiments of an A-B-A block copolymer. Among the three examples, sample-3 shows the fastest degradation rate with the steepest molecular weight drop and the fastest mass loss. The degradation of A-B-A block copolymers depends on A-block constituent type (GA/LA), and the overall weight ratio of the A-block constituent, including its ratio in the B-block.

### Additional Terms/Definitions

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

Following are some further example embodiments of the invention. These are presented only by way of example and are not intended to limit the scope of the invention in any way. Further, any example embodiment can be combined with one or more of the example embodiments.

Embodiment 1. A block copolymer comprising polyglycolide (PGA) or poly-L-lactide, forming an A block and a random copolymer of glycolide (GA), L-lactide, DL-lactide, trimethylene carbonate (TMC), and-caprolactone (CL) forming a B block.

Embodiment 2. The block copolymer of embodiment 1, wherein the block copolymer is an ABA tri-block copolymer with A blocks and an intervening B block.

Embodiment 3. The block copolymer of any of embodiment 1-2, wherein each A block has a weight % ranging from about 10% to about 35%, or about 15% to about 25%, by weight of the block copolymer and, in the case of the ABA tri-block copolymer, the A blocks together have a weight % ranging from about 20% to about 70%, or about 30% to about 50%, by weight of the block copolymer.

Embodiment 4. The block copolymer of any of embodiment 1-3, wherein the B block has a weight % ranging from about 30% to about 80%, or about 50% to about 70%, by weight of the block copolymer.

Embodiment 5. The block copolymer of any of embodiment 1-4, wherein the block copolymer comprises glycolide (GA), trimethylene carbonate (TMC), and ε-caprolactone (CL) monomers in a weight ratio of about 50/25/25 (GA:TMC:CL), in a weight ratio of about 60/20/20 (GA:TMC:CL), or in a weight ratio of about 50-60 parts GA:10-40 parts TMC:10-40 parts CL, with the proviso that the block copolymer includes 100 parts of combined GA, TMC and CL.

Embodiment 6. The block copolymer of any of embodiment 1-5, wherein the B block comprises glycolide (GA), trimethylene carbonate (TMC), and-caprolactone (CL) monomers in a weight ratio of about 15/20/20 (GA:TMC:CL), a weight ratio of about Oct. 20, 2020 (GA:TMC:CL), a weight ratio of about 15/25/25 (GA:TMC:CL), a weight ratio of about 30/20/20 (GA:TMC:CL), or in a weight ratio of about 15-30 parts GA:10-40 parts TMC:10-40 parts CL.

Embodiment 7. The block copolymer of any of embodiment 1-6, wherein the block copolymer can have an inherent viscosity in a range of about 0.5 to about 1.8 dL/g, about 0.7 to about 1.5 dL/g, or about 0.9 to about 1.1dL/g.

Embodiment 8. The block copolymer of any of embodiment 1-7, wherein the block copolymer has a first glass transition temperature (Tg-1) of an amorphous phase in a range of about −30° C. to about 0° C., or about −20° C. to about −5° C., and a second glass transition temperature (Tg-2) of the amorphous phase in a range of about 30° C. to about 55° C., or about 30° C. to about 40° C.

Embodiment 9. The block copolymer of any of embodiment 1-8, wherein the Tm can be in a range of about 110° C. to about 200° C., or about 145° C. to about 200° C., and ΔH can be in a range of about 5 J/g to about 50 J/g, or about 10 J/g to about 35 J/g.

Embodiment 10. The block copolymer of any of embodiment 1-9, wherein, the block copolymer has an enthalpy of fusion (ΔH) in a range of about 5 J/g to about 35 J/g, or about 13 J/g to about 30 J/g.

Embodiment 11. The block copolymer of any of embodiment 1-10, wherein the block copolymer can have a percent crystallinity in a range of about 5% to about 25%, about 5% to about 20%, or about 7% to about 15%.

Embodiment 12. The block copolymer of any of embodiment 1-11, wherein the block copolymer has a tensile strength in a range of about 5 MPa to about 100 MPa, or about 10 MPa to about 40 MPa, or about 10 MPa to about 30 MPa.

Embodiment 13. The block copolymer of any of embodiment 1-12, wherein the block copolymer has an elongation at break in a range of about 50% to about 1000%, about 100% to about 800%, or about 200% to about 400%.

Embodiment 14. The block copolymer of any of embodiment 1-13, wherein the block copolymer can have an elastic modulus in a range of about 20 MPa to about 500 MPa, preferably about 40 MPa to about 400 MPa, and more preferably about 40 MPa to about 200 MPa.

Embodiment 15. An implantable device comprising an implantable device body; and a block copolymer as in any of embodiments 1-14 on and/or forming the implantable device body.

Embodiment 16. The implantable device of embodiment 15, wherein the implantable device body comprises a stent having a stent thickness ranging from about 50 um to about 200 μm.

Embodiment 17. The implantable device of embodiment 15, wherein the stent comprises a plurality of interstices and a thickness of the polymeric material within each interstice is about 50 microns when in an expanded state.

Embodiment 18. The implantable device of any of embodiments 15-17, wherein the implantable device body comprises a vessel closure device.

Embodiment 19. The implantable device of any of embodiments 15-18, wherein one or more layers of the block copolymer is configured to be electrospun onto the implantable device body.

Embodiment 20. The implantable device of any of claims 15-19, wherein the block copolymer is an ABA tri-block copolymer with A blocks and an intervening B block, wherein each A block has a weight % ranging from about 10% to about 35%, or about 15% to about 25%, by weight of the block copolymer and the A blocks together have a weight % ranging from about 20% to about 70%, or about 30% to about 50%, by weight of the block copolymer.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A block copolymer comprising:
at least one of polyglycolide (PGA) or poly-L-lactide (PLLA) forming an A block; and
a random copolymer of at least one of glycolide (GA), L-lactide (LLA) or DL-lactide (DLLA), and trimethylene carbonate (TMC), and ε-caprolactone (CL) forming a B block.

2. The block copolymer of claim 1, wherein the block copolymer is an ABA tri-block copolymer with A blocks and an intervening B block.

3. The block copolymer of claim 1, wherein each A block has a weight % ranging from about 10% to about 35%, or about 15% to about 25%, by weight of the block copolymer and, in the case of an ABA tri-block copolymer, the A blocks together have a weight % ranging from about 20% to about 70%, or about 30% to about 50%, by weight of the block copolymer.

4. The block copolymer of claim 1, wherein the B block has a weight % ranging from about 30% to about 80%, or about 50% to about 70%, by weight of the block copolymer.

5. The block copolymer of claim 1, wherein the block copolymer comprises glycolide (GA), trimethylene carbonate (TMC), and ε-caprolactone (CL) monomers in a weight ratio of about 50/25/25 (GA:TMC:CL), in a weight ratio of about 60/20/20 (GA:TMC:CL), or in a weight ratio of about 50-60 parts GA:10-40 parts TMC:10-40 parts CL, with the proviso that the block copolymer includes 100 parts of combined GA, TMC and CL.

6. The block copolymer of claim 1, wherein the B block comprises glycolide (GA), trimethylene carbonate (TMC), and ε-caprolactone (CL) monomers in a weight ratio of about 15/20/20 (GA:TMC:CL), a weight ratio of about Oct. 20, 2020 (GA:TMC:CL), a weight ratio of about 15/25/25 (GA:TMC:CL), a weight ratio of about 30/20/20 (GA:TMC:CL), or in a weight ratio of about 15-30 parts GA:10-40 parts TMC:10-40 parts CL.

7. The block copolymer of claim 1, wherein the block copolymer has an inherent viscosity in a range of about 0.8 dL/g to about 1.8 dL/g, about 0.9 dL/g to about 1.3 dL/g, or about 0.9 dL/g to about 1.1 dL/g.

8. The block copolymer of claim 1, wherein the block copolymer has a first glass transition temperature (Tg-1) of an amorphous phase in a range of about −30° C. to about 0° C., or about −20° C. to about −5° C., and a second glass transition temperature (Tg-2) of the amorphous phase in a range of about 30° C. to about 55° C., or about 30° C. to about 40° C.

9. The block copolymer of claim 1, wherein the block copolymer has a melting temperature (Tm) of a crystalline phase in a range of about 110° C. to about 200° C., or about 145° C. to about 200° C., and an enthalpy of fusion (ΔH) in a range of about 5 J/g to about 50 J/g, or about 10 J/g to about 35 J/g.

10. The block copolymer of claim 1, wherein, the block copolymer has an enthalpy of fusion (ΔH) in a range of about 5 J/g to about 35 J/g, or about 13 J/g to about 30 J/g.

11. The block copolymer of claim 1, wherein the block copolymer has a percent crystallinity in a range of about 5% to about 25%, about 5% to about 20%, or about 7% to about 15%.

12. The block copolymer of claim 1, wherein the block copolymer has a tensile strength in a range of about 5 MPa to about 100 MPa, or about 10 MPa to about 40 MPa, or about 10 MPa to about 30 MPa.

13. The block copolymer of claim 1, wherein the block copolymer has an elongation at break in a range of about 50% to about 1000%, about 100% to about 800%, or about 400% to about 600%.

14. The block copolymer of claim 1, wherein the block copolymer has an elastic modulus of about 20 MPa to about 500 MPa.

15. An implantable device comprising:
an implantable device body; and
a block copolymer as in claim 1 on and/or forming the implantable device body.

16. The implantable device of claim 15, wherein the implantable device body comprises a stent having a stent thickness ranging from about 50 μm to about 200 μm.

17. The implantable device of claim 16, wherein the stent comprises a plurality of interstices and a thickness of the polymeric material within each interstice is about 50 microns when in an expanded state.

18. The implantable device of claim 15, wherein the implantable device body comprises a vessel closure device.

19. The implantable device of claim 15, wherein one or more layers of the block copolymer is configured to be electrospun onto the implantable device body.

20. The implantable device of claim 15, wherein one or more layers of the block copolymer is configured to be applied through spraying, roller-coating or ink-jetting.

21. The implantable device of claim 20, wherein one or more layers of the block copolymer include an active pharmaceutical ingredient.

22. The implantable device of claim 15, wherein the block copolymer is an ABA tri-block copolymer with A blocks and an intervening B block, wherein each A block has a weight % ranging from about 10% to about 35%, or about 15% to about 25%, by weight of the block copolymer and the A blocks together have a weight % ranging from about 20% to about 70%, or about 30% to about 50%, by weight of the block copolymer.
